(19) United States
(12) Patent Application Publication
(10) Pub. No.: US 2009/0169588 A1
(43) Pub. Date: Jul. 2, 2009

(54) Compositions Containing Proteins for the Transfer/Recycling of Structurally Modified Lipids, and the Applications Thereof

(75) Inventors: Rolf D. Beutler, Höchst/ Hummetroth (DE); Karlheinz Schmidt, Gomaringen (DE)

Correspondence Address: COLLARD & ROE, P.C., 1077 NORTHERN BOULEVARD, ROSLYN, NY, 11576, US

(73) Assignee: Merz Pharma GmbH & Co. KGaA

(21) Appl. No.: 12/085,095

(22) PCT Filed: Oct. 24, 2006
(86) PCT No.: PCT/EP2006/010223

§ 371 Date: May 15, 2008

(30) Foreign Application Priority Data
Nov. 28, 2005 (DE) 102005056538.7

(51) Int. Cl. A61K 9/127; A61K 38/02; A61K 38/44; A61K 36/82; A61K 36/45

(52) U.S. Cl. 424/401; 514/2; 424/94.4; 424/729; 424/732

(57) ABSTRACT

The invention relates to compositions containing recycling/transfer proteins for structurally/functionally modified lipids, a lipid depot of structurally non-modified lipids, and non-transferable active ingredients. Such compositions can be used for the engineering of biomembranes, e.g. by external topical application, especially to the skin/mucous membranes. Structurally or functionally, especially oxidatively modified, lipids are removed from the structures containing the same, such as lipid aggregates of the skin, are externally regenerated in a depot, and preferably introduced back into the structures. The invention also relates to the recycling of modified lipids of biomembranes. The inventive compositions are self-regulative and can be especially applied in a cosmetic, cosmetic balneological or dermatological/pharmaceutical manner.

### SUBJECT MATTER OF THE INVENTION

The present invention relates to compositions containing recycling-transfer proteins for structurally/functionally modified lipids, a lipid depot of structurally unmodified lipids and non-transferable active ingredients. Such compositions can be used for engineering of biomembranes, for example by topical application, especially on skin/mucous membranes. In the process, lipids that have been modified structurally or functionally, above all oxidatively, are removed from the structures containing them, such as lipid aggregates of the skin, regenerated externally in a depot and preferably returned to the structures. This therefore represents recycling of modified lipids from biomembranes. In this regard the compositions are self-regulating, and can be used above all for cosmetic, cosmetic-balneological or else dermatological/pharmaceutical purposes.

### BACKGROUND OF THE INVENTION

Lipids have great structural and functional importance as components of technical products, essential commodities, regular and luxury foods, pharmaceuticals, diagnostics, cosmetics, hygiene and medicinal products. Lipids also have a special role in biological materials, where they are used among other purposes as energy carriers, thermal and electrical insulators, diffusion-evaporation barriers, signal substances, coloring agents, protective agents, enzyme cofactors or else structure-imparting elements of organelles, cells, organs and organisms.

Numerous lipid classes are known to those skilled in the art, examples being fatty acids, fatty alcohols, ester lipids, ether lipids and steroids. These structurally different lipid classes can have different physical and chemical properties. For example, amphiphilic lipids such as the phospholipids, which are widely distributed in biological material, have the property that, in the presence of water, they readily form aggregates, clusters or phases (micelles, monolayers, double layers, multilayers, liposomes, tubes) with very specific structural and functional properties. Other substances such as proteins, carbohydrates, other biopolymers or man-made polymers can also participate in the formation of higher aggregates from amphiphilic lipids, and can additionally modify the properties. The lipid aggregates present in biological materials and also in essential foods, pharmaceuticals or man-made products contain unsaturated lipids, which are important for the function of the aggregates. However, these can be readily oxidized by atmospheric oxygen, UV light, chemical oxidants and free radicals. An example is the “rancidization” of unsaturated edible fats. Such oxidation frequently takes place in radical chain reactions, which usually cease only when the substrate has been consumed. Other structural and thus functional changes or impairments of lipids are possible, such as isomerization, formation of higher aggregates, oligomerization and polymerization. It is not uncommon for the reaction or oxidation products formed during modification of lipids, especially by oxidative modification, to be toxic, above all in the organism. The oxidation of lipid aggregates of biological membranes is therefore also related to the process of biological aging, in which some end products of oxidation, such as ceroids or lipofuscins, are deposited in the tissue and are referred to as age pigments.

Heretofore attempts to prevent lipid modifications, especially due to oxidation, have been made by supplying the lipid-containing structures with substances having an anti-oxidative effect. These include, for example, phenolic substances, which become anchored in the lipid aggregates by means of lipophilic isoprene groups and upon oxidative attack are themselves transformed to the quinoid form (see also German Patent 199 62 369 A1). The protection against lipid modification becomes greater as the concentration of anti-oxidative substances becomes higher. According to German Patent 38 15 473 C1, the problem of effective incorporation of lipophilic substances in cell aggregates can be solved by means of what are known as transfer proteins. This system is also used according to German Patent 103 24 256 A1, in order to achieve incorporation or exchange of lipids in the structures containing them by topical application on the skin without the need for undesired emulsifier components. In this connection, the existing lipids are not functionally modified themselves but are supplemented or replaced by other lipids. Thus what is involved here is the transfer of lipids that have not been modified structurally/functionally or oxidatively. As an example vitamin E can be incorporated as a lipophilic anti-oxidative substance in lipid aggregates. For moderate oxidative stresses, moderate concentrations of such anti-oxidants can build up some protection against oxidative aging. In the case of severe oxidative stresses and higher concentrations of anti-oxidants, however, the oxidized secondary products accumulate in the lipid aggregates and can themselves lead to functional disorders there. By analogy, the aforesaid transfer proteins for unmodified lipids can also be used, according to German Patent 10 2004 057 150 A1, together with a capillary active system. According to German Patent 693 00 514 T2, certain cholesterol-reduced and therefore transparent vesicle dispersions are proposed for transport of active ingredients. In German Patent 694 00 746 T2, combinations of two lipid dispersions are used as active-ingredient carrier for the treatment of both external and internal skin layers. According to German Patent 697 30 604 T2, oxa diacids are used to treat skin disorders, such as aged skin. According to German Patent 698 13 935 T2, pigmentation disorders such as in aged skin should be treated by means of retinoids containing phenol groups.

### OBJECT OF THE PRESENT INVENTION

The object of the present invention is therefore to remedy the shortcomings described hereinabove and to provide a system with which the structural and functional impairment of lipid aggregates can be effectively countered, without causing accumulation of undesired harmful secondary or reaction products.

### ACHIEVEMENT OF THE OBJECT

This object is achieved according to the invention by compositions containing
a) a depot comprising one or more structurally/functionally unmodified lipids;
b) one or more recycling-transfer proteins with transfer rates for structurally/functionally modified lipids;
c) one or more non-transferable active ingredients for regeneration of structurally/functionally modified lipids;
d) if necessary, one or more application-related adjuvants.

The recycling-transfer proteins are in particular those with transfer rates for oxidatively modified lipids and the active ingredients are those for regeneration of oxidatively modified lipids, such lipids being above all of biological origin. The active ingredients for regeneration, above all of oxidatively modified lipids, are not transferable, and so accumulation of secondary products derived therefrom in the lipid structures or aggregates can be avoided. Thus the structurally and above all oxidatively modified lipid, especially of biological origin, can be extracted from the aggregates containing it by means of the transfer protein provided for the purpose, transferred into the depot together with unmodified, above all oxidatively unmodified lipids, regenerated by the active ingredients present here and then transferred back. Surprisingly, an unexpected increase of efficiency of the measures directed against the structural modification, above all oxidation, of lipids takes place in this respect due to the external regeneration. The energy barrier that exists between the two compartments, or in other words the lipid aggregate containing structurally modified lipids on the one hand and the depot on the other hand, and that hinders exchange via the equilibrium imposed on structurally modified lipids by the thermodynamic driving force, can be overcome by means of the transfer proteins.

Particularly suitable compositions contain transfer proteins or hydrolyzed proteins of the described type from animal, plant or marine sources, and possibly also from microbiological sources, such as grains, tubers, milk, silk, having the features as explained hereinafter.

In a further embodiment, transfer proteins having transfer activity for structurally/functionally unmodified lipids according to the prior art (German Patent 38 15 473 C1 or German Patent 103 24 256 A1) are additionally used, leading to increased catalytic transfer of unmodified lipids.

The lipids are selected mainly from natural or synthetic fats, oils, waxes, fatty alcohols, fatty acid esters, fatty acid partial esters, glycerides, silicone oils, silicone waxes, hydrocarbons, lecithins, sphingolipids, cholesterols, phospholipids, gangliosides, cerebrosides, ceramides or mixtures thereof. Very particularly suitable as the lipid depot is a fatty phase in the form of unilamellar or multilamellar liposomes, which are derived from the cited substances, namely lecithins, sphingolipids, phospholipids, ceramides or mixtures thereof.

Active ingredients that are particularly suitable for recycling are above all non-transferable, especially water-soluble or water-dispersible active ingredients such as relatively high molecular weight enzyme proteins (such as peroxidases), cofactors such as glutathione, alpha-liponic acid, water-soluble anti-oxidants such as ubiquinone, and redox systems such as erythorbic acid.

Furthermore, plant-based phenols such as quercetin, apple quercetin, cystus tea extract (from Cystus incanus ssp. Tauricus) can be used as non-transferable active ingredients. Particularly preferred as non-transferable active ingredients are peroxidases, erythorbic acid and glutathione as well as cystus tea extract, apple quercetin or mixtures thereof.

Preferably the compositions contain adjuvants that are suitable for the form of application, preferably topical application, and that are selected from water and additives, including in particular surface-active substances (such as oil-in-water and water-in-oil co-emulsifiers, co-surfactants or mixtures), colloids of natural or synthetic origin, consistency regulators, coloring agents, perfumes, preservatives, skin-care and additional active ingredients or mixtures thereof.

### MORE DETAILED DESCRIPTION OF THE COMPOSITIONS

The inventive compositions are designed for topical application or administration on mucous membranes, and they comprise a fat or oil phase containing suitable lipids for the lipid depot, one or more recycling proteins suitable for transfer of modified lipids, one or more non-transferable active ingredients suitable for recycling and adjuvants suitable for the desired application. These are preferably selected from medically or pharmaceutically acceptable additives and water. In this respect, selective engineering of biomembranes in vivo, ex vivo or in vitro can be achieved, as can that of synthetically produced lipid membranes. Administration can take place in particular for cosmetic, dermatological, pharmaceutical or cosmetic-balneological purposes, using a form of application suitable for the respective case. The inventive compositions can preferably have the form of a cream (such as oil-in-water, water-in-oil, gel cream), lotion, gel, balsam, spray, mask or foam. Above all, agents can be provided for the purpose of anti-aging cosmetics, for example by application on the skin or by action on the skin in baths or showers. Furthermore, the agents can be used on mucous membranes, for example for administration in the case of plaques and lesions.

The individual components of the compositions will be explained hereinafter.

### 1) Proteins for Lipid Transfer

Because of the slight water solubility of lipids, the exchange of individual lipid molecules between lipid aggregates in an aqueous medium takes place slowly, or in other words less efficiently, and it does so substantially by virtue of the thermal motion of the molecules. By means of lipid transfer proteins, the activation energy between the lipid aggregates can be catalytically lowered (see also German Patent C138 15 473).

Recycling proteins to be used according to the invention are generally obtained from plant, animal, genetically engineered or possibly microbial sources, and on the whole have an average molecular weight of approximately 2500 to 40,000 D, depending on origin. In this connection, plant proteins from grains such as oats, millet, wheat, corn, barley and spelt have an average molecular weight of 4000 to 8000 D, proteins from tubers or fruits have an average molecular weight of 6000 to 20,000 D, proteins from milk have an average molecular weight of 2500 to 8000 D, proteins from silk, microorganisms and marine sources have an average molecular weight of 10,000 D to 40,000 D, above all from 15,000 D to 40,000 D. Mixtures may also be used. The cited proteins can also be produced by genetic engineering.

Suitable as tubers are potatoes, ylang ylang, topinambur or similar species. Examples of marine sources are algae, mussels and seafood. Examples of suitable microorganisms are bacteria. Proteins of plant and animal origin as well as proteins from marine sources are preferred, although the former are particularly preferred. Among marine products, algal proteins are particularly preferred, above all those with an average molecular weight of 12,000 to 25,000 D.

Plant proteins are in particular those from grains, especially spelt, millet, oats, corn and soy, while animal proteins are above all those from milk and silk.

Particularly preferred proteins are selected from plant proteins, especially oats, wheat, corn, barley, soy, beans, millet, spelt, buckwheat, tapioca, topinambur and potatoes or mixtures thereof. Especially preferred are millet, spelt, buckwheat, tapioca; or else oats, corn, barley or wheat; or mixtures thereof.

The proteins or hydrolyzed products suitable for transfer can be obtained by slurrying in water, if necessary reaction with acids or bases, further processing such as centrifuging, if necessary separation of accompanying substances by precipitation, for example with acid or salts (such as ammonium sulfate), defatting with organic solvents, and purification by dialysis, gel filtration or chromatography, etc. Methods for this purpose are described in the prior art, and especially in German Patent 38 15 473 C1; see also U.S. Pat. Nos. A 5,776,470 and A 6,077,529.

The proteins used according to the invention have a transfer rate for structurally modified, especially oxidatively modified lipids. This transfer rate can be determined in the resonance energy transfer test (RET test); see Nichols, J. W., Pagano, R. E., J. Biol. Chem. 258 (1983), 5368-5371. For this purpose there can be used, as lipid aggregates, liposomes obtained in the conventional way (for example by detergent dialysis and size determination by scattered-light measurement) and having unsaturated lipids, such as 10% dioleylphosphatidylcholine, which carry two different fluorescence markers, such as N—NBD (N-[7-nitro-2,1,3-benzoxadiazol-4-yl] and N-Rh(N-[Lissamine Rhodamine B]). The lipids are oxidized at the double bonds by known methods, such as with atmospheric oxygen and a radical chain initiator such as 1,1′-azobis(cyclohexanecarboxylic acid nitrile). The degree of oxidation can be determined in a known way such as thin-layer chromatography of the phospholipids or, after hydrolysis, by gas chromatography of the fatty acid methyl esters. The catalytic activity of the proteins is determined on the basis of the transfer, brought about by the proteins being used, of the fluorescence-marked lipids from the donor liposomes to the acceptor liposomes.

Surprisingly, it has been found that the inventive systems are active when the specific activity of the recycling-transfer proteins for the cited, especially oxidized lipids is between 10 and 10,000 ng/min/mg of protein, especially 150 to 10,000 and preferably 500 to 9000 and especially 800 to 8000 ng/min/mg of protein.

Particularly preferred are inventive recycling-transfer proteins for modified lipids that have a transfer rate as indicated above, preferably 600 to 8000 ng/min/mg of protein, and an average molecular weight of 3500 to 8000 D, and that are derived from plant proteins, particularly grains, and in this category especially oats, wheat, corn, barley, millet, spelt; or from soy, potatoes or topinambur, with an average molecular weight of 6000 D to 15,000 D. A further preferred group of transfer proteins that are suitable according to the invention are those from milk, with an average molecular weight of 2500 to 8000 D, or from silk, with an average molecular weight of 15,000 to 20,000, respectively with a transfer rate as indicated, preferably from 500 to 7000 ng/min/mg of protein.

Mixtures of the aforesaid preferred plant and animal proteins are also particularly suitable.

Proteins from marine sources and having the cited suitable transfer rates can also be selected, especially from algae, mussels and corals. Their average molecular weight is in particular 7500 D, and the transfer rate in this case is preferably 300 to 8500 ng/min/mg of protein.

Proteins obtained by microbiological methods are derived, for example, from fungal cultures, and they have the said transfer rate as well as an average molecular weight of 15,000 D to 25,000 D.

Very particularly preferred are plant proteins such as described, animal proteins from silk or above all from milk as described hereinabove. Mixtures of proteins from these two groups are also particularly suitable. In particular, the proteins used according to the invention are at least water-dispersible, but above all are water-soluble. They function above all as a transfer vehicle. They can be used in proportions of 0.01 to 10 wt %, above all 0.01 to 8 wt %.

Preferably there are selected proportions of 0.01 to 5 wt %, especially 0.01 to 3 wt %, above all 0.01 to 0.95%, and very particularly 0.01 to 0.85 or else 0.1 to 0.85, especially 0.1 to 0.5 wt %.

Combinations of representatives from the various groups can also be used, especially from animal proteins and plant proteins of the cited kind. In this connection, the ratio of the substances from the different groups can be varied, for example from 1:4 to 1:10, especially 1:1 to 1:6. Suitable above all is milk protein, especially hydrolyzed milk protein in combination with a plant protein, selected from oats protein, barley protein, millet protein or spelt protein as described, above all with preferred molecular weights, or marine proteins in the respectively indicated, particularly preferred range of transfer rates and molecular weights.

In some cases, certain recycling proteins of the described type having the indicated molecular weights and transfer rates additionally also catalyze the transfer of unmodified lipids, such as those from millet, buckwheat, spelt and tapioca (especially if they have the preferred average molecular weights and transfer rates indicated in the foregoing), and this may lead to a further unexpected improvement of the system by accelerated transfer of unmodified or regenerated lipids. These are therefore also preferred. According to the invention, there are also contained, in a further preferred embodiment, further proteins having transfer rates for lipids that have not been structurally, especially oxidatively modified, as described in the prior art (German Patent 38 15 473 C1 or German Patent 103 24 256 A1), and this then leads to further catalytically activated transfer of unmodified lipids, above all regenerated lipids, into the corresponding aggregates. In this category there belong proteins from oats, wheat, corn (average molecular weight 3800 to 7700, especially 5800 to 7700), milk (average molecular weight 2700 to 6800, for example), seafood (average molecular weight 11,500 to 23,000) or mixtures thereof, as described especially in German Patent A1103 24 256). On the whole, therefore, recycling takes place. The transfer activity of such proteins for lipids that have not been modified structurally, especially oxidatively, can be determined in the known way; see German Patent 103 24 256 A1.

2. Lipid depot

Structurally unmodified lipids are used to provide an inventive lipid depot.

To this category there belong in particular lecithins such as soy and egg lecithin, sphingolipids and phospholipids, which also represent vesicle-forming agents. By means of the latter agents, the formation of unilamellar or multilamellar liposomes is possible either inherently, for example in the presence of suitable surfactant additives and water, or externally in a known way such as high-pressure homogenization, an ultrasonic method, an extrusion method or detergent dialysis. Micellar emulsions are obtained from polar lipids.

Further common fats, oils or waxes that contribute to absorption of the non-transferable active ingredient can also be used as the lipid depot. In this category there belong above all hydrocarbons such as squalene, squalane and especially also liquid paraffins, isoparaffins, dioctylcyclohexanes, isodecane and isohexadecanes.

Also suitable are natural or synthetic fats, oils, waxes, polysiloxane compounds, triglycerides or monoglycerides/diglycerides of C₁₂₋₂₂ fatty acids, C₈₋₂₂ fatty alcohols, such as oleyl alcohol, octyldodecanol, cetyl/stearyl alcohol; C₁₂₋₂₂ fatty acid partial esters of polyhydric C₂₋₆ alcohols, such as especially monoglycerides, or diglycerides, for example glycerol monostearate/distearate and mixtures thereof; polyol C₁₂₋₂₂ fatty acid esters, such as PEG-7-glyceryl cocoate, propylene glycol dicaprylate/dicaprate, mixtures such as hexyldecanol and hexyldecyl laurate, and C₁₂₋₂₂ fatty acid partial esters of C₂₋₆ alcohols. Fatty acid esters such as C₂₋₁₈ alcohol fatty acid esters, such as isopropyl fatty acid esters (palmitate, myristate, isostearate and oleate), decyl oleate, hexyl laurate, C12-15 alkyl benzoates and dicaprylyl carbonates as well as branched fatty acid esters such as cetearyl octanoate and di-n-butyl adipate are also suitable. Also usable are C₈₋₂₂ fatty alcohol ethers such as dicaprylyl ether or fatty alcohol esters such as C₁₂₋₁₃ alkyl lactates, especially glycerides (C₁₂₋₂₂ fatty acid glycerides) such as triglycerides, especially medium-chain (neutral oils) such as caprylic/capric triglycerides, as well as their polyol esters such as propylene glycol dicaprylate/dicaprate.

Natural fats, oils and waxes include, for example, sunflower, soy, peach-kernel, apricot-kernel, grapeseed, castor, olive, peanut, almond, mink, wheat-germ and avocado oil, shea butter or illipe butter, natural liquid waxes, such as jojoba oil or its substitute, oleyl erucate, natural beeswax and similar substances.

Examples of synthetic or semisynthetic waxes are bleached beeswax, Kester® Wax K82H(C₂₀₋₄₀ alkyl stearate) or Lunacera® M (Micro Wax) or hydrocarbon waxes such as Lunacera® P (Mineral Wax) as well as hydrogenated castor oil (Cutina® HR) or synthetic waxes such as cetyl palmitate (Cutina® CP) or myristyl myristate (Crodamol® MM), or stearyl stearate (Crodamol® SS).

Examples of polysiloxane compounds are silicone oil waxes such as polydimethylsiloxanes (dimethicone), cyclomethylsiloxanes (cyclopentasiloxanes), phenylmethylpolysiloxanes such as phenyl dimethicone or alkylpolymethylsiloxane copolymers such as cetyl dimethicone and stearyl dimethicone, dialkoxydimethylpolysiloxanes such as stearoxy dimethicone and behenoxy dimethicone, which can be used in particular with other lipids mentioned hereinabove.

As mentioned, the lipids are used in particular as the liposomal phase or else, if the other lipid substances are being used alone or in combination with vesicle-forming substances, as emulsions, especially micellar emulsions. For this purpose there are used suitable additives, such as mentioned hereinafter, especially water and surface-active substances, and the mixture of lipid and additive is converted to the desired emulsions, for example by stirring and/or homogenizing, in suitable devices.

Besides the vesicle-forming lipids, especially phospholipids, lecithins such as soy and egg lecithin, phosphatidylcholine, phosphatidylserine, phosphatidylethanolamine, phosphatidylglycerol, phosphatidylinositol, gangliosides, cerebrosides, cholesterol and ceramides, such as ceramide-2, 3 and 6 as well as sphingolipids, other substances suitable as the lipid depot are in particular natural oils, fats and waxes as mentioned, triglycerides, C₈₋₂₂ fatty alcohols; C₁₂₋₂₂ fatty acid partial esters of polyhydric C₂₋₆ alcohols.

Simple stable emulsions such as oil-in-water and water-in-oil emulsions can also be produced as described hereinafter, using suitable additives.

Preferred are micellar emulsions from polar lipids, as well as lipid depots from phospholipids, lecithins such as soy and egg lecithin, phosphatidylcholine, phosphatidylserine, phosphatidylethanolamine, phosphatidylglycerol, phosphatidylinositol, gangliosides, cerebrosides, cholesterol and ceramides, such as ceramide-2, 3 and 6 as well as sphingolipids.

In another suitable embodiment, lamellar lipid depots such as described can also be used in combination with natural and/or synthetic lipids, especially triglycerides, saturated, unsaturated and partly saturated C₆₋₂₄ fatty alcohols, C₈₋₃₀ fatty acid esters, above all the natural representatives of the cited groups, especially natural oils and waxes such as sunflower oil, olive oil, peach-kernel oil, wheat-germ oil, orange oil, almond, rape, soy and coconut oil, plant waxes, fruit waxes and beeswax.

The lipid depot proportion is guided by the respectively desired form of application, and is preferably 1 to 70 wt %, especially 3 to 60 wt % and above all 8 to 57 wt %. If lotions, sprays or gel-like products are desired, the lipid proportion is preferably 5 to 45 wt %. Especially for balneological purposes, for example, the content as a bath additive can be between 25 and 70 wt % of lipids and as a shower additive can preferably be 5 to 35 wt % of lipids. For formulations in the form of a fatty cream (such as water-in-oil), the content can be especially 5 to 60 wt % of lipid depot.

### 3. Non-Transferable Active Ingredients

The non-transferable active ingredients include in particular those that can restore structurally modified lipids to their basic structure by interaction therewith. The following substances in particular belong to this category:

relatively high molecular weight enzyme proteins such as peroxidases, cofactors such as glutathione and analogous sulfhydryl compounds, antioxidants such as ubiquinones, plant phenolic compounds such as cystus tea extract, apple quercetin, redox systems such as erythorbic acid or mixtures thereof. These active ingredients, above all peroxidases, cofactors such as glutathione, cystus tea extract, apple quercetin and erythorbic acid, are particularly suitable for recycling oxidized lipids.

Furthermore, there can also be used water-soluble or at least water-dispersible non-transferable active ingredients, selected from ascorbic acid, α-liponic acid, NADH, NADPH, green-tea extract or mixtures thereof.

Also suitable are water-soluble extracts from blueberries or elderberries or else from grapes or strawberries or mixtures thereof.

Also possible are rutine and ferula acid.

Particularly preferred are peroxidases, glutathione, analogous sulfhydryl compounds and ubiquinones.

These active ingredients are in particular water-dispersible to water-soluble, and are incorporated into the lipid depot by production methods known in themselves. For this purpose the aqueous phase is prepared first, in this case of the desired recycling active ingredient or ingredients, then the lipid depot phase is prepared and the two phases are then mixed with one another using suitable known apparatuses and if necessary also further additives such as colloids, surface-active agents, consistency regulators, etc. The non-transferable active ingredients are present in proportions known for the application, such as 0.01 to 9 wt %, especially 0.1 to 7% and preferably 0.01 to 5 wt %, depending on the system.

### 4. Adjuvants

The type of adjuvants is guided by the desired form of application, and it includes water in particular as well as additives.

The additives are selected above all from surface-active agents, colloids, skin-care agents, consistency regulators, additional active ingredients, preservatives, perfumes, coloring agents or mixtures thereof.

Surface-active agents are emulsifiers or dispersion agents, and they can be contained in proportions of 0.1 to 20 wt %, preferably 0.1 to 10 wt %, especially 1 to 7 wt %. They include water-in-oil dispersing agents such as preferably sorbitan derivatives (sorbitan oleate, sorbitan stearate), ethoxylated fatty acids/alcohols/esters such as PEG2/4 stearate, ceteareth 3, (poly)glyceryl esters (such as polyglyceryl-3-stearate), glycerol esters such as glyceryl ricinoleate, polymers such as polyoxypropylene-polyoxyethylene block polymers, polysiloxane copolymers such as polysiloxane-polyether copolymers, especially polysiloxane-polyalkyl-polyether copolymers such as cetyl dimethicone copolyol, polyvalent salts such as magnesium, aluminum or zinc stearate, triglycerides such as ethoxylated C₈₋₂₂ fatty alcohol/C₁₂₋₂₂ fatty acids, C₁₂₋₂₂ fatty acid monoesters and diesters of addition products of ethylene oxide and C₃₋₆ polyols, ethoxylated alkylglycosides, (poly)glyceryl derivatives, polyol esters, pentaerythritol derivatives, alkylphenols or mixtures thereof, and very particularly among those Abil® EM 90, Arlacel ®582 and magnesium stearate or mixtures thereof. Co-emulsifiers such as stearyl-cetearyl alcohol, stearic acid, and also Ariatone®T(V) can also be present here. Further examples of surface-active agents are oil-in-water emulsifiers such as in particular polyoxyethylated products such as those of the magrogol type (PEG fatty acids/alcohols/fatty acid esters with a chain length of 6 to 22 C atoms in the fatty acid/ester part and degrees of ethoxylation of 5 to 30), nonionic and ionic phosphates, ionic univalent salts, sterol derivatives, castor oil derivatives, siloxanes or mixtures thereof or mixtures containing co-emulsifiers thereof. Suitable as oil-in-water emulsifiers are in particular polyoxyethylated products, nonionic and ionic phosphates, ionic univalent salts, (poly)glyceryl esters, sterol derivatives, castor oil derivatives, siloxanes or mixtures thereof or mixtures containing co-emulsifiers thereof. Particularly suitable here are Tego Care® 450, Emulgin® B1 or mixtures thereof and/or containing co-emulsifiers. By analogy, there are particularly preferred, as water-in-oil emulsifiers, sorbitan derivatives, polyethoxylated fatty acid/alcohol/esters/triglycerides, (poly)glycerol derivatives, polyol esters, glucose derivatives, pentaerythritol derivatives, alkylphenols, (block) polymers, fatty acid salts, siloxanes or mixtures thereof, and very particularly among those Abil® EM 90, Arlacel® 582 and magnesium stearate or mixtures thereof.

Co-emulsifiers such as Arlatone®T(V) can also be present here.

Depending on degree of ethoxylation and/or alkyl chain length, either oil-in-water or water-in-oil dispersing agents are contained. Accordingly, a higher degree of ethoxylation and/or short chain length lead(s) to oil-in-water products, while the opposite characteristics lead to water-in-oil products. This is known to those skilled in the art, and so suitable products can be chosen on the basis of the corresponding HLB value (2 to 7=water-in-oil, 8 to 18=oil-in-water, limit range 9 to 13 for water dispersibility). Thus the desired emulsions can be obtained, for example, by combination of suitable products.

Furthermore, (co)-surfactants are also possible as surface-active agents, especially anionic and nonionic surfactants, which can also be present in particular for applications with additional cleaning function. As nonionic surfactants of this category there belong above all alkoxylated fatty acid esters, for example of the formula R1CO(OCH2CHR2)ₓCR3, where R1CO=linear, branched, saturated and/or unsaturated C₆₋₂₂ acyl groups, R2=H or methyl, R3=C1-4 alkyl, x=1 to 20 and similar products (such as glycerides).

Suitable as anionic surfactants are glutamates, such as sodium cocoyl glutamate (Hostapon® CCG) or else such products selected from alkyl sarcosinates, alkyl sulfates, alkyl ether sulfates, alkyl sulfosuccinates, alkyl sulfosuccinamates, isethionates or their alkali salts or mixtures thereof. These have in particular 8 to 22, preferably 8 to 16 carbon atoms in the alkyl chain. Particularly preferred here are alkyl ether sulfates derived from fatty alcohols having 12 to 18 carbon atoms and a degree of ethoxylation of 2 to 6, such as the Na salt of lauryl/myristyl ether sulfate, ammonium lauryl ether sulfate or monoisopropanolammonium lauryl ether sulfate or alkyl sulfates, such as sodium lauryl sulfate, ammonium lauryl sulfate or monoisopropanolammonium lauryl sulfate.

Very particularly preferred are alkyl sarcosinates such as sodium lauroyl, cocoyl or oleyl sarcosinates, succinic acid derivatives such as alkyl sulfosuccinates and alkyl sulfosuccinamates containing an alkyl group of 8 to 22 carbon atoms or mixtures thereof. Above all, the cited alkyl sarcosinates are preferred.

Emulsions, especially emulsion gels, can preferably also be obtained by the use of colloids.

Colloids (preferably 0.01 to 8%, for example) are especially high or relatively high molecular weight substances of natural or (semi)synthetic origin or combinations thereof such as high molecular weight plant proteins having a molecular weight of 100,000 D and higher, relatively high molecular weight proteins such as caseinates, for example having an average molecular weight of 18,000 and higher, gelatins or synthetic polymers such as acrylate polymers having high molecular weight (0.2 to 3 million) as well as copolymers thereof, especially after neutralization by alkali. Preferred here are the polyacrylate Carbopol® types known under the INCI name of carbomers, such as Carbopol® 910, 934, 940, 941, 954, 980, 981, 2984, 5984 or Carbopol® ETD 2001, 2050 or Synthalen® K, L, M or the already neutralized carbomers such as PNC® 400, 410, 430 (INCI: sodium carbomer). There can also be used acrylate copolymers, such as arylates/C10-30 alkyl acrylate cross polymers, known as Carbopol® 1342, 1382, ETD 2020, Pemulen® TR-1, TR-2.

Further suitable colloids are acrylamides as well as polysaccharides such as Keltrol® (xanthan gum), cellulose derivatives such as hydroxypropyl methyl cellulose (Methocel® J12-MS) or ethylcellulose or silicates such as magnesium aluminum silicate (Veegum® HV), in each case having high molecular weight, especially higher than 50,000.

Preferred acrylamides are polyacrylamide, such as Fiocare® T 920 GC, possibly polyacrylamide-containing mixtures such as Sepigel® 305 (polyacrylamides, C13-14 isoparaffins, laureth-7) Sepigel® 501 (acrylamides copolymer, mineral oil, C13-14 isoparaffins, polysorbate 85), Sepigel® 502 (C13-14 isoparaffins, isostearyl isostearate, sodium polyacrylates, polyacrylamides, polysorbate 20), Creagel® EZ DC (polyacrylamide, polydecene, dimethicone copolyol), Creagel® EZ 5 (polyacrylamide, polydecene, laureth-5).

Colloids are preferably used together with co-emulsifiers such as the fatty alcohols cited hereinafter under consistency regulators.

Examples of skin-care agents (preferably 0.01 to 20%, for example, especially 0.01 to 10%) are phytosterols (substantially mixtures of β-sitosterol, campesterol and stigmasterol) as well as those comprising rape oil (Generol® R), silicone (co)polymers, such as Dow Corning® HMW 2220 (divinyldimethicone/dimethicone copolymer, C12-13 pareth-3/C12-13 pareth-23). Further skin-care agents are moisturizers such as glycerol, propylene glycol or polyethylene glycols, propylene glycol, butylene glycol, sorbitol or polymers, such as polyquaternium types such as polyquaternium-39 (Merquat® plus 3330), amino acids, urea, polysaccharides such as Fucogel® 1000 (biosaccharide gum-1), glucosaminoglycans, such as hyaluronic acid or sulfated glucosaminoglycans such as chondroitic sulfate, dermatan sulfate, keratan sulfate, heparan sulfate, especially the Na salts thereof or heparin Na, glucans, such as β-glucan, for example β-glucan from oats (Drago-β-glucan®), mannans such as Konjac Mannane, or commercial moisturizers such as Hydractin® (glycerol, water, disodium adenosine triphosphate, algin, carica papaya) or Aquaderm® (sodium PCA), sodium lactate, fructose, glycines, niacinamide, urea, inositol), salts, such as sodium lactate, and the Na salt of DL-2-pyrrolidone-5-carboxylic acid.

Preferred are moisturizers such as polyethylene/propylene glycol or glycerol in proportions such as 0.5 to 10%, especially 5 to 10%, as well as polysaccharide compounds such as Fucogel® 1000, and/or the Na salt of hyaluronic acid.

Examples of consistency regulators are complexing agents such as Trilon®BD (EDTA, Na salt), means for adjusting pH, such as citric acid, caustic soda, solvents such as propylene glycol or alcohols, starches or starch derivatives, starches such as rice, wheat, corn and potato starches, hydrophobically modified starches such as Dry Flo® AF (modified cornstarch), aluminum starch octenyl succinate (Dry Flo® PC, Fluidamid® DF 12), hydroxypropyl starch phosphate esters (Structure® XL) or mixtures thereof such as Natrasorb® HFB (aluminum starch octenyl succinate, acrylates copolymer, magnesium carbonate), ASO/MM3® (aluminum starch octenyl succinate, magnesium myristate), Dry Flo® Elite LL (aluminum starch octenyl succinate, lauroyl lysine), Facemat® (aluminum starch octenyl succinate, mica, Zea Mays (Com) starch, silica, titanium dioxide, zinc oxide), mixtures of the cited substances thereof.

Further suitable consistency regulators are, for example, fatty alcohols, such as stearyl alcohol (Lanette® 18), cetyl alcohol (Lanette® 16), myristyl alcohol (Lanette® 14) or cetearyl alcohol (Lanette® 0). Also suitable are glyceryl esters such as glyceryl stearate, especially glycerol monostearate or glycerol distearate or mixtures thereof, such as Tegin® M. If appropriate, these can also be interpreted as co-emulsifiers.

The inventive compositions are in particular neutral to the skin and gentle on the skin or mucous membranes, and in this regard they preferably have a pH of ≦7.

In addition, one or more preservatives (preferably 0.01 to 5% each, for example) may also be present. Examples of suitable preservatives are iodopropynylbutyl carbamate, DMDM hydantoin, phenoxyethanol and further common preservatives, such as sorbic and dehydroacetic acid and salts thereof, methyl dibromoglutanonitrile, etc., or combinations thereof, or other acids such as benzoic or salicylic acid, or benzyl alcohol or esters such as p-hydroxybenzoic acid esters, for example methyl, ethyl, propyl, butyl or isobutyl paraben, preferably methyl or propyl paraben, or mixtures thereof or climbazol or suitable combinations of the cited substances.

Examples of suitable perfumes (preferably 0.01 to 5%, for example) are the ethereal oils cited under active ingredients or commercial perfume mixtures.

As coloring agents (preferably 0.01 to 2%, for example) there are preferably chosen the components known for the products of the class in question, such as Patent Blue, Amido Blue, Orange RGL, Cochenille Red and Quinoline Yellow.

Additional active ingredients (0.01 to 10%, for example) are in particular vitamins, plant extracts, synthetically produced substances corresponding to these extracts and analogous derivatives thereof, as well as skin-influencing active ingredients and mixtures thereof.

Examples of plant extracts are those obtained from ylang ylang, ginkgo, pine needles, cypress, birch-leaf extract, aloe-vera extract, marigold, hibiscus, burdock, witch hazel, march pennywort leaf, algal, quince, water lily and cinnamon extracts, extracts from thyme, mint, limes, oranges, grapefruit, mandarins, juniper, valerian, lemon balm, eucalyptus, thyme, palmarosa, rosemary, lavender, rosewood, lemon grass, wild rose, spruce needles and pine needles, ginger, red-currant, lime-blossom, marigold, magnolia, pineapple, guava, Echinacea and ivy-leaf extracts or mixtures thereof.

These extracts can be produced, for example, by steam distillation. Hereby there are obtained from the cited plants, for example, ethereal oils, which are particularly preferred.

The extracts can also be obtained in other known ways, for example by solvent extraction (with alcohols, triglycerides or hydrocarbons, water and mixtures thereof) and then used as such. Examples of analogous synthetically produced substances are terpenes and terpenoids such as camphor, menthol, cineol or mixtures thereof.

Examples of suitable vitamins are vitamin A, E or other or suitable derivatives thereof, such as esters, for example palmitate, acetate or phosphate.

Also suitable are mineral substances and trace elements such as copper, zinc or derivatives thereof such as Zincidone® (zinc PCA), zinc gluconate or copper gluconate.

There can also be used astringent and sebum-regulating substances such as Acnacidol® 101 (propylene glycol, hydroxydecanoic acid), Asebiol® BT (hydrolyzed yeast protein, pyridoxine, niacinamide, glycerol, panthenol, propylene glycol, allatoin, biotin), Lipacide® C8C0 (caproyl collagen amino acids), Sebosoft® (glycerol, water, PEG-8, caprylyl glycol, sebacic acid, sodium polyacrylate), Sepi Control A5 (capryloylglycine, methylglycine, cinnamonum zeylanicum).

Also usable as additional active ingredients are blood-circulation-promoting substances, for example nicotinic acid derivatives such as methyl or tocopheryl nicotinate, alpha and beta hydroxyl acids and derivatives thereof, for example glycolic, malic, citric, tartaric and lactic acids, salicylic acid, isopropylbenzyl salicylate, C12-13 alkyl lactates (Cosmacol® ELI) or else anti-inflammatory and antibacterial substances such as triterpenes, for example ursolic acid, glycyrrhizinic acid or glycyrrhetinic acid and derivatives thereof, for example stearyl glycyrrhetinates, potassium glycyrrhinate, pantothenic acid derivatives, for example D-panthenol, panthenyl triacetate; allatoin, bisabolol; azulenes, such as chamazulene or guajazulene; phytosphingosines; triclosan; chlorhexidine derivatives and/or anti-dandruff agents, such as climbazol or piroctone olamine.

Substances having cytoprotective effects, such as coenzyme Q 10, can also be used as active adjuvants in the inventive compositions.

Further additional active ingredients are UV filters such as UVB, UVA and broadband filters of the following type:

UVB filters: cinnamic acid esters, such as ethylhexyl methoxycinnamate, isoamyl p-methoxycinnamate as well as 4-methylbenzylidine camphor, paraminobenzoic acid and esters such as N,N-dimethyl-4-aminobenzoic acid 2-ethylhexyl ester, homomethyl salicylate, octyl salicylate, octocrylene phenylbenzimidazolesulfonic acid or benzylidene malonate polysiloxane, UVA+UVB filters for broadband absorption such as benzophenone-3 (Neo® Heliopan BB, Eusolex® 4360).

UVA filters such as methyl anthranilate, butyl methoxydibenzoylmethane, methylene bis-benzotriazolyl tetramethylbutylphenol, bis-ethylhexyloxyphenol methoxyphenyltriazine, disodium phenyl dibenzimidazole tetrasulfonate 2-(4-diethylamino-2-hydroxybenzoyl)benzoic acid hexyl ester.

Particularly preferred additional active ingredients are ethereal oils or extracts of ylang ylang, lemon grass, mint, oranges, wild rose, grapefruit, limes, ginkgo, mandarins, cypress, thyme, lavender, rosemary, ginger or mixtures thereof.

Furthermore, there are also above all used the said vitamins and/or panthenol, especially in combination with one or more plant extracts/ethereal oils.

Particularly desired additional active ingredients are selected from vitamins, UV filters, plant extracts or else dermatological active ingredients, for example antimycotics such as naftifin HCl, terbinafin HCl or antioxidative (above all in small proportions), antibacterial and/or sebum-regulating substances.

The type and proportion of additives are guided by the desired form of application and the desired purpose. Preferably at most 40%, especially 0.01 to 30%, above all also 0.01 to 25%, preferably 0.1 to 20% or 1 to 18%, especially 5 to 15% of additives are present. Particularly preferably there are selected as adjuvants, besides water, consistency regulators, coloring agents, perfumes, colloids, surface-active (interface-active) agents, skin-care agents, possibly preservatives or mixtures thereof, while skin-care agents, consistency regulators and additional active ingredients are particularly preferred.

Particularly preferred are adjuvants selected from water and 0.01 to 30%, especially 0.1 to 20 wt % of additives, which above all are selected from additional active ingredients, skin-care agents, coloring agents, perfumes, surface-active agents, especially colloids, co-surfactants, consistency regulators and preservatives.

Preferred compositions are characterized in that they contain 0.01 to 10 wt % of one or more transfer proteins b), 1 to 70 wt %, preferably 1 to 50 wt %, especially 1 to 40 wt % of the lipid depot a), 0.01 to 10 wt % of one or more non-transferable active ingredients c) and d) 1 to 30 wt %, above all 1 to 20 wt % of additives, especially selected from additional active ingredients, skin-care agents, coloring agents, perfumes, surface-active (interface-active) agents as indicated, colloids, consistency regulators, preservatives or mixtures thereof as well as water (as the balance).

In a further embodiment, there are contained 0.05 to 5 wt %, especially 0.1 to 3 wt % of transfer protein b), 1 to 60 wt %, above all 5 to 53 wt % of lipid depot a), 0.05 to 5 wt %, especially 0.1 to 5 wt % of active ingredient c), and d) 1 to 25 wt %, preferably 1 to 20, especially 1 to 15 wt % of additives, selected from additional active ingredients, skin-care agents, coloring agents, perfumes, surface-active (interface-active) agents such as oil-in-water, water-in-oil, co-emulsifiers, co-surfactants, colloids, consistency regulators, preservatives or mixtures thereof as well as water (as the balance).

5. Forms of application:

Depending on water content, which can range between 5 and 80 wt %, the following (exemplary) forms of application may be contained (in each case with additions cited by way of example):

gels (for example, 50 to 80% water) containing colloids, co-emulsifiers and optionally additional active ingredients, skin-care agents, coloring agents, perfumes, consistency regulators or mixtures thereof;
oil-in-water or water-in-oil creams (for example, 20 to 70% water) containing surface-active agents/colloids and optionally additional active ingredients, skin-care agents, coloring agents, perfumes, consistency regulators or mixtures thereof;
balsam (for example, 10 to 60% water): containing surface-active agents/colloids and optionally additional active ingredients, skin-care agents, coloring agents, perfumes, consistency regulators or mixtures thereof;
bath or shower additives (for example, 30 to 80% water) containing colloids, co-surfactants, co-emulsifiers and optionally additional active ingredients, skin-care agents, coloring agents, perfumes, consistency regulators or mixtures thereof.
FUNCTIONAL principle of the recycling system

The inventive compositions contain a lipid depot, special proteins and special active ingredients as characterized for catalytic transfer or recycling of structurally modified lipids. These active ingredients are incorporated but not transferred into the lipid depot. In this way the thermodynamic driving force for transfer of modified lipids is preserved, and so the process is not exhausted by equilibrium distribution in the two compartments (lipid depot without/lipid aggregate containing structurally modified lipids), as is the case of the systems described in the prior art. It is therefore possible to control the instant of the duration of action by concentrating the non-transferred active ingredient or ingredients. As an example, this can be achieved with appropriate forms of application, such as night cream with high active ingredient contents, milk (such as cleansing milk), bath additives with medium active ingredient proportions and shower preparations with somewhat smaller active ingredient proportions. Recycling of the modified lipids then takes place outside the lipid-aggregate structures, and so unphysiological accumulations of active ingredients cannot occur. Thereby undesired effects on these structures are avoided.

After recycling, the recycled lipids are returned to the aggregate simultaneously according to the invention or else additionally in the presence of the corresponding transfer proteins according to the prior art, such as German Patent 38 15 473 C1.

The following net effect is achieved: Independently of the specificity of catalytic transfer of the modified lipids into the inventive depot, the modified lipids are unilaterally removed from the aggregates by recycling outside the aggregate structures (remote recycling), since no net effect takes place with respect to the unmodified lipids, because the forward and reverse transfer thereof is unchanged. In the case of given specificity of the catalytic transfer for modified lipids, the net transfer for the modified lipids from the aggregates still depends only on the concentration of the modified lipids. If the content thereof in the lipid aggregate increases, so also does the content thereof transferred into the depot increase.

The inventive system for recycling of modified, especially oxidatively modified lipids from lipid aggregates thus has self-regulating properties, in that the efficiency of recycling increases to the same extent as the modifications to the lipid aggregates become larger. At the same time, the physiological composition of the aggregates is largely preserved, due to the unchanged forward and reverse transfer of unmodified lipids, and so no undesired effects can occur. The inventive recycling-transfer proteins can therefore be employed or used for transfer/recycling of structurally, especially oxidatively modified lipids, above all by topical application on the skin/mucous membranes, or else for engineering of biomembranes. In addition, they can also be used in particular in systems or compositions that are suitable for the purpose or even in compositions of components a), b), c) and d), such as described hereinabove for the production thereof. Such systems or compositions are suitable above all for topical application, for example in the form of a cream, gel, lotion, spray, mask, balsam, shower or bath additive, balsam, milk, for transfer/recycling of structurally, especially modified lipids, for example of the skin or else of the mucous membranes, for example with respect to the indications cited hereinafter, especially for skin and hair.

### Administration of the Recycling System

Depending on the intended purpose, the inventive compositions can also be used, by selection of suitable adjuvants, especially for cosmetic, cosmetic-balneological or else dermatological/therapeutic administration on skin, hair or mucous membranes. In this regard the compositions can have the form of cream, lotion, milk, gel, gel emulsions, milk, balsam, spray, shower additive, bath additive (foaming if necessary) or mask, and can be used as such for lipid-regenerating skin care or cleansing in cosmetic or cosmetic-balneological applications. This results in improvement of the appearance of irritated, aged, especially light-damaged, dry sensitive skin or hair, or in other words of functionally/structurally and above all oxidatively altered skin/mucous membranes. Particularly preferred is administration in the form of a cream or gel cream as well as a shower or bath additive or else as a mask. By means of the inventive combination, the transfer proteins used can develop their effect as a vehicle for modified lipophilic/amphiphilic components. Due to the transfer of modified lipids, external recycling and return transport, undesired substances do not remain in the lipid structures. Thus regeneration of oxidatively modified lipids, especially for cosmetic purposes, can be achieved as described by application of an inventive composition on the surface and above all on the skin of humans. Further effects can be achieved by choice of suitable additional active ingredients. Administration can also be used for dermatological/pharmaceutical purposes. Above all, these involve inflamed conditions, irritated conditions of the skin, for example as a result of allergy, or skin burns, as well as for irritated conditions of the respiratory tract, for example as a result of allergy, or irritations of potential application areas. In this regard, it is possible by topical external administration of the inventive compositions to regenerate modified and above all oxidatively modified lipid aggregate structures, above all if they are influenced by aging, damaged, especially light-damaged, dry, sensitive, inflamed skin of mammals, especially humans.

### Production of the Products

The inventive compositions are produced by preparing the lipid phase by mixing and if necessary heating the desired lipid or lipids and possibly also by separate production of liposomes if desired, as described. In this case soluble additives can be incorporated therein. Water-soluble or water-dispersible constituents such as additives, active ingredients of group c) as well as protein(s) of group b) are incorporated in the desired concentration, and the two phases are intermixed at suitable temperatures such as 20 to 80° C., preferably 30 to 60° C. to room temperature, in such a way that a stable composition in the form of an emulsion/gel/dispersion, etc. is obtained. After cooling, thermally stable substances can be added while stirring if necessary. This conversion is carried out in such a way that, on the one hand, high dispersity of the coherent phase is achieved, so that the non-transferable active ingredients remain in the depot, and, on the other hand, the integrity of the transfer proteins is preserved.

### EXAMPLES

The invention will be explained in more detail on the basis of the examples hereinafter (quantitative data refer to percent by weight unless otherwise indicated):

### Example 1

### Composition on a Water-In-Oil Basis

| | |
|---|---|
| Fatty alcohol ether (Cetiol ® OE) | 5.00 |
| Apricot-kernel oil | 5.00 |
| Beeswax | 5.00 |
| Evening primrose oil | 2.00 |
| Oxynex 2004 | 0.05 |
| Propylene glycol | 4.00 |
| D-Panthenol | 5.50 |
| Hydrolyzed barley protein | 0.80 |
| (average MW: approximately 5500) | |
| Glutathione | 1.50 |
| Sorbitan isostearate (Arlacel ®582) | 7.00 |
| PEG40 sorbitan peroleate (Arlatone TV) | 1.50 |
| Magnesium stearate | 2.00 |
| Coloring agent (pigment paste) | 2.00 |
| Perfume oil (Deliana) | 0.50 |
| Water | made up to 100 |

### Example 2

### Composition on an Oil-In-Water Basis

| | |
|---|---|
| Soy lecithin (Phosal SA 50) | 2.00 |
| Jojoba oil | 1.00 |
| Beeswax | 5.00 |
| Evening primrose oil | 2.00 |
| Micro wax (Lunacera ® M) | 2.50 |
| Fatty alcohol ether (Cosmacol ®ELI) | 3.00 |
| Hydrolyzed milk protein | 0.50 |
| (average MW 3000) | |
| Algal protein (average MW | 0.50 |
| approximately 18,000) | |
| Sodium chloride | 0.50 |
| Polyglyceryl-3-methyl glucose distearate | 3.00 |
| (Tego Care ®450, oil-in-water) | |
| Carbopol ETD 2020 | 0.25 |
| Hydrogenated castor oil (Cutina ® HR) | 0.40 |
| Propylene glycol | 4.00 |
| Marigold extract | 2.00 |
| Deliana perfume oil | 0.50 |
| Cystus tea extract | 2.00 |
| Water | made up to 100 |

### Example 3

### Oil-In-Water Gel Emulsion

| | |
|---|---|
| Avocado oil | 3.0 |
| Stearyl alcohol | 2.0 |
| Glyceryl stearate | 2.0 |
| Isopropyl stearate | 6.0 |
| Dicaprylyl ether | 3.0 |
| Squalane | 5.0 |
| Phosphatidylcholine | 1.0 |
| Hydrolyzed millet protein | 0.8 |
| (average MW approximately 4500) | |
| Hydrolyzed milk protein | 0.6 |
| (average MW approximately 3500) | |
| Butylene glycol | 2.0 |
| Disodium EDTA | 0.1 |
| Sodium caseinate | 3.0 |
| (MW approximately 20,000) | |
| Cyclomethicone | 1.0 |
| Preservatives | sufficient quantity |
| Perfume | 2.0 |
| Apple quercetin | 0.4 |
| Water | made up to 100 |

### Example 4

### Oil-In-Water Cleansing Emulsion

| | |
|---|---|
| Almond oil | 2.0 |
| Cetyl alcohol | 2.0 |
| C12-15 alkyl benzoates | 3.0 |
| Squalane | 1.0 |
| Cetearyl isononanoate | 2.0 |
| Algal protein (average MW 15,000) | 1.5 |
| Acrylates/C10-30 alkyl acrylates | 0.2 |
| Cross polymer | |
| Hydroxypropylethylcellulose | 0.2 |
| Glycerol | 3.0 |
| Disodium EDTA | 0.1 |
| Sodium lauryl sarcosinate | 2.0 |
| Triethanolamine | 0.25 |
| Preservative(s) | sufficient quantity |
| Perfume | 0.04 |
| α-Liponic acid | 1.00 |
| Water | made up to 100 |

### Example 5

### Bath Additive

| | |
|---|---|
| Soy oil | 10.00 |
| Jojoba oil | 5.00 |
| Isopropyl myristate | 10.00 |
| Lecithin | 5.00 |
| Hydrolyzed spelt protein | 1.20 |
| (average MW 4500-5000) | |
| Laureth 4 | 7.00 |
| Polyoxyethylene(7) glyceryl acetate | 5.00 |
| Panthenol | 3.00 |
| Eucalyptus oil | 2.50 |
| Perfume oil | 0.80 |
| Blueberry extract | 3.00 |
| Water | made up to 100 |

### Example 6

### Shower Additive

| | |
|---|---|
| Olive oil | 3.00 |
| Almond oil | 1.50 |
| Sphingosine | 1.50 |
| Hydrolyzed milk protein | 1.00 |
| (MW 6000) | |
| Silk protein (MW 12,000) | 0.50 |
| Xanthan gum | 0.50 |
| Hydroxypropylmethylcellulose | 1.50 |
| Cystus tea extract | 1.50 |
| Water | made up to 100 |

1. Compositions containing:
a) a depot comprising one or more structurally/functionally unmodified lipids;
b) one or more recycling-transfer proteins with transfer rates for structurally modified lipids;
c) one or more non-transferable active ingredients for regeneration of structurally/functionally modified lipids;
d) if necessary, one or more application-related adjuvants.

2. Compositions according to claim 1, wherein the structurally/functionally modified lipids are oxidatively modified lipids and the lipids in the lipid depot are oxidatively unmodified lipid(s).

3. Compositions according to claim 1, wherein the recycling-transfer protein(s) have transfer rates for oxidatively modified lipids.

4. Compositions according to claim 1, wherein they further contain one or more transfer proteins having transfer rates for lipids that have not been structurally/functionally, especially oxidatively modified.

5. Compositions according to claim 1, wherein the lipid or lipids of depot a) is or are selected from natural or synthetic fats, oils, waxes, fatty alcohols, fatty acid esters, fatty acid partial esters, glycerides, silicone oils, silicone waxes, hydrocarbons, lecithins, sphingolipids, cholesterols, phospholipids, gangliosides, cerebrosides, ceramides or mixtures thereof.

6. Compositions according to claim 5, wherein they contain the lipid depot in the form of liposomal lipid structures, comprising unilamellar or multilamellar liposomes.

7. Compositions according to claim 1, wherein the recycling transfer protein or proteins b) is or are selected from animal, plant and marine proteins or mixtures thereof.

8. Compositions according to claim 7, wherein the transfer protein or proteins b) is or are selected from grains having an average molecular weight of 4000 to 8000 D, tubers or fruits having an average molecular weight of 6000 to 20,000 D, proteins from milk having an average molecular weight of 2500 to 8000 D, or proteins from silk and marine sources having an average molecular weight of 10,000 D to 40,000 D, or mixtures thereof.

9. Compositions according to claim 8, wherein the recycling-transfer protein or proteins has or have a transfer rate of 10 to 10,000 ng/min/mg of protein for structurally/functionally modified and especially oxidized lipids.

10. Compositions according to claim 8, wherein the recycling-transfer protein or proteins for structurally/functionally modified and especially oxidized lipids has or have a transfer rate of 600 to 8000 ng/min/mg of protein and an average molecular weight of 3500 to 8000 D, and is or are derived from plant proteins, especially oats, wheat, corn, barley, soy, beans, millet, spelt, buckwheat, tapioca, topinambur, potatoes.

11. Compositions according to claim 10, wherein the recycling-transfer protein or proteins is or are selected from proteins from buckwheat, millet, spelt, tapioca.

12. Compositions according to claim 8, wherein the recycling-transfer protein or proteins for structurally/functionally modified and especially oxidized lipids is or are that or those from milk, with an average molecular weight of 2500 to 8000 D, or from silk, with an average molecular weight of 15,000 to 20,000, respectively with a transfer rate as indicated, preferably from 500 to 7000 ng/min/mg of protein.

13. Compositions according to claim 8, wherein they contain mixtures of plant and animal proteins according to claim 10 to 12 as the transfer protein(s).

14. Compositions according to claim 1, wherein active ingredient or ingredients c) is or are selected from water-soluble or water-dispersible active ingredients of the group comprising erythorbic acid, glutathione, alpha-liponic acid, plant phenol compounds, relatively high molecular weight enzyme proteins, cofactors, water-soluble anti-oxidants and redox systems.

15. Compositions according to claim 14, wherein the non-transferable active ingredient c) is selected from NADH, peroxidases, erythorbic acid, glutathione, alpha-liponic acid, glutathione, green-tea extract, cystus-tea extract, apple quercetin, blueberry extract, elderberry extract or mixtures thereof.

16. Compositions according to claim 14, wherein the non-transferable active ingredient c) is selected from erythorbic acid, glutathione, cystus-tea extract, apple quercetin or mixtures thereof.

17. Compositions according to claim 1, wherein they contain 0.01 to 10 wt % of one or more recycling-transfer proteins b), 1 to 40 wt % of the lipid depot a), 0.01 to 10 wt % of one or more active ingredients c) and d) adjuvants selected from 0.1 to 20 wt % of additives as well as water (as the balance).

18. Compositions according to claim 1, wherein they contain adjuvants for topical application.

19. Compositions according to claim 1, wherein they contain, as adjuvants, one or more substances selected from water and additives selected from colloids, consistency regulators, surface-active agents, preservatives, adjuvants, perfumes, coloring agents, skin-care agents or mixtures thereof.

20. Compositions according to claim 19, wherein the surface-active agents are selected from oil-in-water emulsifiers, water-in-oil emulsifiers, co-emulsifiers, co-surfactants or mixtures thereof.

21. Compositions according to claim 19, wherein they exist in the form of a liquid, semiliquid or solid substance as an oil-in-water emulsion, water-in-oil emulsion, gel emulsion, gel, cream, balsam, lotion, milk, facial mask, spray, bath additive or shower additive.

22. Method for regeneration of oxidatively modified lipid aggregates, wherein a composition according to claim 1 is brought into contact with the lipid aggregates to be treated by application on the surface.

23. Method according to claim 22, wherein, by application of a composition, oxidatively modified lipids are exported from the lipid aggregates containing them, recycled in depot a) by means of one or more active ingredients c) and then transferred back into the lipid aggregate.

24. Method according to claim 22, wherein as regards the lipid aggregates, it relates to the skin of humans and to cosmetic regeneration.

25. Use of compositions according to claim 1 for topical cosmetic or cosmetic-balneological regenerative skin care or regenerative gentle cleansing of the skin of mammals, especially of humans.

26. Use of compositions according to claim 1 for production of a means for preventive, dermatological, therapeutic regenerative treatment of the skin of mammals.

27. Use of compositions according to claim 1 for production of a means for selective engineering of biomembranes in vivo.

28. Use of compositions according to claim 1 for selective engineering of biomembranes ex vivo, in vitro.

29. Use of compositions according to claim 1 for selective engineering of synthetically produced lipid membranes.

30. Compositions containing:
a) a depot comprising one or more structurally/functionally unmodified lipids, selected from natural or synthetic fats, oils, waxes, fatty alcohols, fatty acid esters, fatty acid partial esters, glycerides, silicone oils, silicone waxes, hydrocarbons, lecithins, sphingolipids, cholesterols, phospholipids, gangliosides, cerebrosides, ceramides or mixtures thereof;
b) one or more recycling-transfer proteins with transfer rates for structurally/functionally modified and especially oxidized lipids, which protein or proteins has or have a transfer rate of 600 to 8000 ng/min/mg of protein and an average molecular weight of 3500 to 8000 D, and is or are derived from proteins from buckwheat, millet, spelt, tapioca;
c) one or more non-transferable active ingredients for regeneration of structurally/functionally modified lipids, selected from water-soluble or water-dispersible active ingredients of the group comprising erythorbic acid, glutathione, alpha-liponic acid, plant phenol compounds, relatively high molecular weight enzyme proteins, cofactors, water-soluble anti-oxidants and redox systems;
d) if necessary, one or more application-related adjuvants.
